(19) United States
(12) Patent Application Publication
(10) Pub. No.: US 2021/0401610 A1
(43) Pub. Date: Dec. 30, 2021

(54) LUMBAR PROTECTION EXOMUSCLE

(71) Applicant: Victor Jose Cainzos Perez, L'Hospitalet de Llobregat (ES)

(72) Inventor: Victor Jose Cainzos Perez, L'Hospitalet de Llobregat (ES)

(21) Appl. No.: 17/473,046

(22) Filed: Sep. 13, 2021

Related U.S. Application Data
(63) Continuation-in-part of application No. PCT/ES2019/070168, filed on Mar. 12, 2019.

(51) Int. Cl. A61F 5/02

(52) U.S. Cl. CPC A61F 5/028; A61F 2005/0197; A61F 5/026

(57) ABSTRACT

A lumbar protection exomuscle is provided that includes of a harness that connects the rear part of the human body, from the shoulders to the feet, held in place, in addition, at the hip and knees, being such points connected successively to each other and in a vertical manner. In a standing position, the device maintains the tension without exerting force, but at the moment of flexion of the hip, the rear area of the thigh and glutes is stretched due to it being to the only elastic zone, thus accumulating a directional force that counteracts the torsions generated in the lumbar area, either at the moment of flexing, in a flexed static position or at the moment of extension.

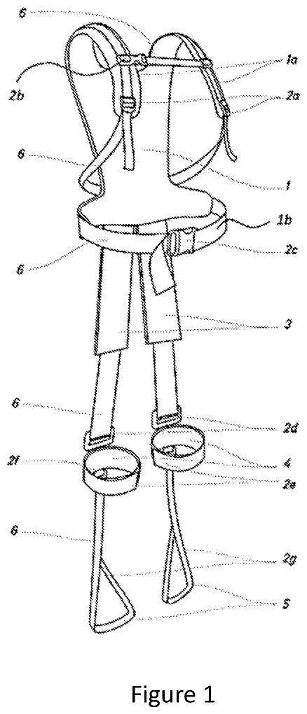

### RELATED APPLICATIONS

This application is a continuation-in-part of International Application No. PCT/ES2019/070168, filed 12 Mar. 2019, the contents of which are hereby incorporated by reference.

### TECHNICAL FIELD

The lumbar exomuscle proposed must be regarded as an orthosis, since the term exoskeleton does not conform to its reality due to de absence of rigid pieces simulating bone structures. Moreover, the purpose of the device is to be an assistive technology oriented to occupational therapy.

### BACKGROUND OF THE INVENTION

As it is known, the spinal column is constituted by a chain of vertebrae linked to each other by intervertebral discs, connected to the pelvis, and this, to the bone system of the lower limbs. This whole structure is assembled by the paravertebral muscles, abdominal muscles, quadratus lumborum, psoas, glutes and pyramidal and femoral biceps, allowing the flexion and extension movement of the whole body.

Continuous movements and efforts of the muscles responsible for the movement of the involved joints influence the health of the anatomy of the lumbar region, such as weight loads, incorrect habits, flexed posture in a continuous way, working conditions, etc.

All these agents influence decisively the appearance of injuries in the musculoskeletal system such as osteoporosis of the vertebrae, muscular contracture, disc rupture or herniation, degeneration of intervertebral discs, misalignment of vertebrae, spinal stenosis, muscle or ligament strain or tear, abnormal spine curvatures, etc. All of these pathologies will cause pain and discomfort that can become chronic and severely affect the activities of the daily life of the user.

In other cases, some genetic factors such as anatomical defects of the spinal column have pathological nature, as in the case of kyphosis, lordosis or scoliosis, said alterations being an added risk for the development of the aforementioned pathologies.

To date, the main assistive technologies used to attempt to reduce the overload of the lumbar area at the bending moments are lumbar braces, since the pressure they exert on the contour of the abdomen limits the movement and the muscular function of the lumbar area. The problem is that this pressure has many drawbacks for the health of the user. In addition, it should be stressed that lumbar braces have not been conceived nor are recommended for said use.

Currently, mechanical artifacts called exoskeletons have been developed, whose function might be the same as that of the lumbar exomuscle, but unlike it, such exoskeletons use rigid structures, machined or not, that beyond their function, cause great drawbacks because they exert pressure over the soft tissues, given the need for support of those rigid structures and the forces they generate on the skeleton of the user, compressing and damaging the soft tissues.

EP1293188 is considered the closest prior art. It shows a exomuscle according to the preamble of claim 1. It comprises elastic foot fasteners.

US2005/130815 or U.S. Pat. No. 6,190,342 are also to be considered, although they disclose artifacts with elastic backpieces.

After these observations, we can state that the claimed lumbar exomuscle performs, in a more efficient and effective manner, the function of the aforementioned products. Aiding the musculoskeletal system at the bending moments, thanks to not limiting muscle mobility as lumbar braces do, but accompanying the muscle in its movement. Moreover, the exomuscle is much lighter, ergonomic and healthy than an exoskeleton, for the simple fact that it is not a rigid structure that uses the body of the user as a lever point.

### SUMMARY OF THE INVENTION

The invention disclosed in the current specification refers to an exomuscle for lumbar protection. It reduces significantly and mainly the force exerted by the lumbar and other muscle groups such as spinal erectors, gluteus maximus and femoral biceps. It achieves so by using a harness which is fastened in different strategic areas such as the shoulders, hip, knees and ankles, being these fasteners connected to each other at the rear part of the body by textile materials, thus creating a vertical structure that traverses the entire rear part of the body (from shoulders to feet), and which is held tight at all times. In addition, these supports effectively distribute the forces generated by the lumbar exomuscle.

The functionality of the system lies in that, between the shoulders and the hip, and the knees and the ankles, the materials are not elastic, whereas between the hip and the knees, an elastic material with vertical elasticity is positioned preferably with a height regulator, being this area the only one that is stretched and deformed at the moment of flexion of the hip. Thus it generates a physical resistance that simulates the muscular function of the muscles responsible for lumbar damping, support and extension.

Because of everything mentioned in the previous section, the demonstrated aid and reduction in the muscle effort by the claimed lumbar exomuscle generate large benefits in preventive aspects of possible musculoskeletal injuries to the lumbar area and, in addition, a significant reduction in the health and social care costs that this type of pathologies generate for the State and the companies themselves.

The invention whose registration is requested corresponds to an orthosis, which we consider as an external muscle (exomuscle) in the form of a harness, which will be described below, together with each of the parts thereof and the relationship between them.

Starting from the rear part of the waist upwards, passing through the rear part of the back, non-elastic fasteners are extended around the shoulders to create the upper support and fastening.

Over the waist area, a support is fastened horizontally around it, thus creating another support point that not only distributes the forces generated, but also prevents lateral displacements of the lumbar exomuscle.

From the support fastened to the waist, elastic deformable materials are extended through the rear part over the gluteus and femoral biceps in vertical direction to the knee fastener. The materials in this area have the function of deforming at the moment when a flexion of the hip occurs, thus creating an elastic tension that simulates the directional force exerted by the muscles of the musculoskeletal system of the aforementioned area.

For securing the elastic deformable materials of the lower end, fasteners are placed horizontally around the knees, specifically on the lower part of the patella. The function of these fasteners is to provide a good anchoring point when the elastic deformable materials generate vertical forces and to maintain them on the rear part of the gluteus and femoral biceps.

Finally, from the knee's fasteners, non-elastic fasteners will extend to the feet area, fastening to them. The function of said fasteners is to subtract tension from the knee's fasteners and to maintain them in a much more comfortable position.

It has also been foreseen that such fasteners may not fit all anatomies, therefore the invention has been provided with the possibility of regulating them to place them at the height and width most suitable in each case. In this regard, it has been arranged that, together with the fasteners of the shoulders, knees and heels, there are adjusting elements that can be regulated both in height and width.

This way, a current problem is solved: a great number of people suffer, or have suffered at some point in their lives, from ailments in the lumbar area caused by an overexertion in said area, since the aforementioned lumbar exomuscle exerts a force aimed to absorb this overexertion of the user's lumbar area, with the consequent decrease of the possibility of injury and palliation of the ailments already existing.

The lumbar protection exomuscle comprises a harness formed by a back piece, two elastic straps and two foot-fasteners. The back piece comprises shoulder straps and a hip strap. The two elastic straps are affixed to the bottom part of the back piece. Each elastic strap comprises a knee fastener for wrapping around the lower area of the patella, or any other relevant area of the user. A foot fastener extends from each elastic strap, stretching to the foot and clasping to the contour of the foot or ankle.

The harness might comprise several regulators that allow for the adaptation in size of each element, mainly the straps or fasteners.

In a preferred embodiment, the foot fastener comprises an element of footwear: shoe or sock.

Elastic material will be understood as any material which can return to its original shape and size after the expected deformation during the use of the exomuscle. It may also comprise a combination of non-elastic and elastic elements.

Assistive technologies comprehend any product including devices, equipment, tools, technology and software that corrects or facilitates the execution of an action, activity or movement, seeking to save energy and achieve greater safety by improving the function of the locomotor system.

### BRIEF DESCRIPTION OF THE DRAWINGS

To complete the description being made and in order to aid a better understanding of the characteristics of the invention, according to an example of a preferred embodiment thereof, as an integral part of the description, a FIGURE is provided for the purposes of illustration only and in a non-limiting manner.

### PREFERRED EMBODIMENT OF THE INVENTION

The lumbar exomuscle in question relates to a harness that comprises elastic textile materials, or of any other type, in its main parts with the exception of the anchors and regulators, which may be of any hard material such as plastic or metal. As shown in the Figure, a back piece (1) is placed at the back and the shoulder straps (1a) that integrate it are intended for the adaptation of the device to the user around the shoulders and hip. The shoulder straps (1a) are not elastic so as to not to detract from the effectivity of the device. The piece (1) is equipped with several regulators (2) that allow for the adaptation in height size with the regulators of the shoulder straps (2a), in chest contour and height with the chest regulator (2b) and in waist contour with the hip regulator (2c) of a hip strap (1b).

Linked to the back piece (1), two elastic straps (3) are joined at the bottom part, either textile or of any other elastic material that accumulates energy in the moment of flexion. The two elastic straps (3) are adjustable in height thanks to the lower regulator (2d) located at their lowest part. To withstand the force generated by the elastic straps (3), knee fasteners (4) are arranged wrapping around the lower area of the patella, or any other relevant area of the user. The knee fasteners (4) are made of textile materials or any other type of material, which allow for comfortable and effective adaptation thereof to the contour of the knees and keep them in place using some type of contour regulator (2e).

In order to avoid the overload caused by the knee fasteners (4), extending therefrom, there are foot fasteners (5) stretching to the foot and clasping to its contour or any other suitable area. Such foot fasteners (5) are made of textile materials or any other type of material whose deformation is minimal so as to not to detract from the effectivity of the device. In addition, the knee fasteners (4) also are adjustable by a height regulator (2f) located in the upper part and by another width regulator (2g) placed around the feet. Finally, each regulator (2) has a strap (6) of textile material or of any other non-elastic type, that allows for a margin of adjustment of the device without causing it to lose effectivity. That is, the strap (6) provides the material for the enlengthening or stores the material when shortening the corresponding part of the exomuscle.

1. A lumbar protection exomuscle that comprises a harness formed by:
a non-elastic back piece with shoulder straps and a hip strap; and
two elastic straps in the bottom part of the back piece
two knee fasteners (4) for wrapping around the lower area of the patella; and characterized in that comprises
two non elastic straps between the two elastic straps and the two knee fasteners two foot fasteners extending from each non elastic straps stretching to the foot and clasping to the contour of the foot or ankle.

2. The lumbar protection exomuscle, according to claim 1, that further comprises several regulators (2) that allow for the adaptation in size.

3. The lumbar protection exomuscle, according to claim 1, where the foot fastener comprises an element of footwear.

4. The lumbar protection exomuscle, according to claim 1, wherein the back piece comprises non-elastic fasteners from the rear part of the waist upwards, passing through the rear part of the back, and extended around the shoulders to create the upper support and fastening.
